(19) United States
(12) Patent Application Publication
(10) Pub. No.: US 2026/0301916 A1
(43) Pub. Date: Oct. 1, 2026

(54) Method and System for Oral Hygiene

(71) Applicant: Oralic Supplies, Inc., Encino, CA (US)

(72) Inventors: Jun Lee, Los Angeles, CA (US); Fang Sun, Seattle, WA (US)

(21) Appl. No.: 19/631,231

(22) Filed: Mar. 27, 2026

Related U.S. Application Data
(60) Provisional application No. 63/779,926, filed on Mar. 28, 2025.

(51) Int. Cl. G16H 20/40; G06T 7/00; G06T 7/11; G16H 10/60; G16H 30/40

(52) U.S. Cl. CPC G16H 20/40; G06T 7/0012; G06T 7/11; G16H 10/60; G16H 30/40; G06T 2207/10016; G06T 2207/20084; G06T 2207/30036

(57) ABSTRACT

Methods and systems for selecting a toothbrush or other oral care product that is particularly effective for a particular person’s dental condition are disclosed. A camera captures an image of the person’s teeth, optionally after the person has chewed an indicator material. The image is processed to determine an oral condition of the person by classifying an image area as corresponding to teeth, identifying a subregion in which plaque or other conditions are present, and using pixel data extracted from the image for the area and the subregion to calculate a distribution of plaque on the teeth. Based on the distribution of plaque and one or more oral characteristics, the system selects, from a data store, an oral care product that corresponds to the distribution of plaque and the characteristics.

### RELATED APPLICATIONS AND CLAIM OF PRIORITY

This patent document claims priority to United States provisional patent application number 63/779,926, filed Mar. 28, 2025. The disclosure of the priority application is fully incorporated into this document by reference.

### BACKGROUND

Brushing teeth is a daily oral care habit of billions of people worldwide. The American Dental Association (ADA) recommends that people brush their teeth at least twice per day for two minutes each time, using a brush with soft bristles. Some people choose manual toothbrushes, while others choose electric toothbrushes. The ADA awards a seal of acceptance to toothbrushes that the ADA has evaluated for safety and efficacy.

However, outside of the basic categories of manual or electric, and stiff or soft, users generally select toothbrushes with brush heads that are similar in effect. While different bristle patterns, colors, stiffnesses, and sizes exist, users typically choose brush heads based on style and/or feel, not based on any assessment of expected clinical effectiveness of the particular brush head. Accordingly, improved methods of selecting toothbrush heads that will be effective for a particular person are needed.

Similar issues exist with other oral care products. Many varieties of toothpaste, dental floss, mouthwashes, teeth whitening products, and other products used for oral hygiene are available. However, there is no easy way for an individual to know which products are most effective for the user’s oral condition. And while dental professionals can help guide users to select appropriate products, that guidance is limited to time when the patient is in the office, as it requires diagnosis and assessment by a medical professional.

This document describes methods and systems that are directed to solving at least some of the issues described above.

### SUMMARY

In various embodiments, this document describes methods and systems for selecting a toothbrush or other oral hygiene product that is particularly effective for a subject’s dental condition.

Various embodiments disclosed in this document are directed to methods and systems for selecting a toothbrush or other oral hygiene product that is particularly effective for a particular person’s dental condition. In some embodiments, an electronic imaging device captures an image of the person’s teeth, optionally after the person has chewed an indicator material such as a plaque disclosing agent. The image is processed to determine an oral condition of the person by classifying an image area as corresponding to teeth, identifying a subregion in which plaque is present, extracting pixel data from the image, and using the pixel data to calculate a distribution of plaque on the teeth. Based on the distribution of plaque and one or more oral characteristics, the system selects, from a data store, an oral care product that corresponds to the distribution of plaque and the characteristics.

Various embodiments also include methods of treating a dental patient using any of the methods described above.

Various embodiments also include computer program products containing programming instructions for implementing any of the methods described above. Various embodiments also include systems that include processors, data stores, and computer program products containing programming instructions for implementing any of the methods described above.

### BRIEF DESCRIPTION OF THE DRAWINGS

FIG. 1 illustrates various aspects of a system that may be used to select an oral hygiene product for a person.

FIGS. 2A and 2B show a flowchart illustrating various steps that the disclosed methods of selecting an oral hygiene product may include.

FIG. 3 illustrates example categories of descriptive information that the system may include in a data store of oral hygiene product information.

FIG. 4 is an image depicting plaque on a person’s teeth.

FIG. 5 illustrates how the system may select an oral care product that is appropriate for a person’s oral characteristics.

FIG. 6 illustrates example features of a user interface of a software application.

FIG. 7 depicts example hardware components that may be included in any of the electronic devices of the system.

### DETAILED DESCRIPTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” (or “comprises”) means “including (or includes), but not limited to.”

In this document, when terms such as “first” and “second” are used to modify a noun, such use is simply intended to distinguish one item from another, and is not intended to require a sequential order unless specifically stated. The term “approximately,” when used in connection with a numeric value, is intended to include values that are close to, but not exactly, the number. For example, in some embodiments, the term “approximately” may include values that are within +/- 10 percent of the value.

Additional terms that are relevant to this disclosure will be defined at the end of this Detailed Description section.

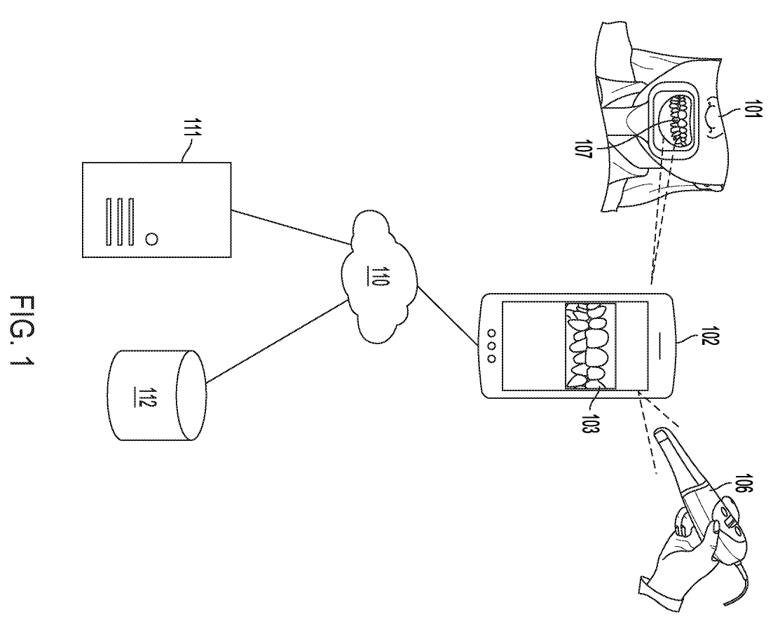

FIG. 1 illustrates various aspects of a system that may be used to select a toothbrush or other oral hygiene product for a person 101. The person 101 may be an individual who is selecting a product for themselves, or the person 101 may be a patient of a dental professional who is selecting the product for, or recommending the product to, the patient. As shown, an electronic device 102 will receive image data 103 for the teeth 107 inside the subject’s mouth 107. The device 102 may use a camera to capture the image data 103 while the subject’s mouth 107 is in the camera’s field of view. In some embodiments, the camera may be a component of the electronic device 102 itself, or of another electronic device. In other embodiments, the camera may be that of a dental scanning device such as a three-dimensional (3D) intraoral scanner in which the image sensor is placed into the person’s mouth and moved around the mouth to capture a 3D image or multiple two-dimensional (2D) images. In other embodiments, the electronic device 102 may include a lidar system, in which case the image data may be a three-dimensional lidar point cloud. Alternatively, the device 102 may receive the images from an electronic device in an electronic message sent via one or more communication networks 110.

The electronic device 102 will include computing device components, or it will be in communication with a remote computing system 111 with such components, that will process the images to analyze the subject’s teeth and recommend an oral hygiene product based on that analysis. The remote electronic device 102 and/or remote computing system 111 may include rules-based programming instructions that are configured to cause the device to process image data, a machine learning model that has been trained to identify certain dental characteristics in images of a mouth and/or teeth, or a combination of these. Methods by which the computing device(s) may do this will be described below. The system also will include a data store 112 made up of one or more memory devices that store data about various oral hygiene products, along with descriptions of each product, associations of each product with a category, and/or other data elements.

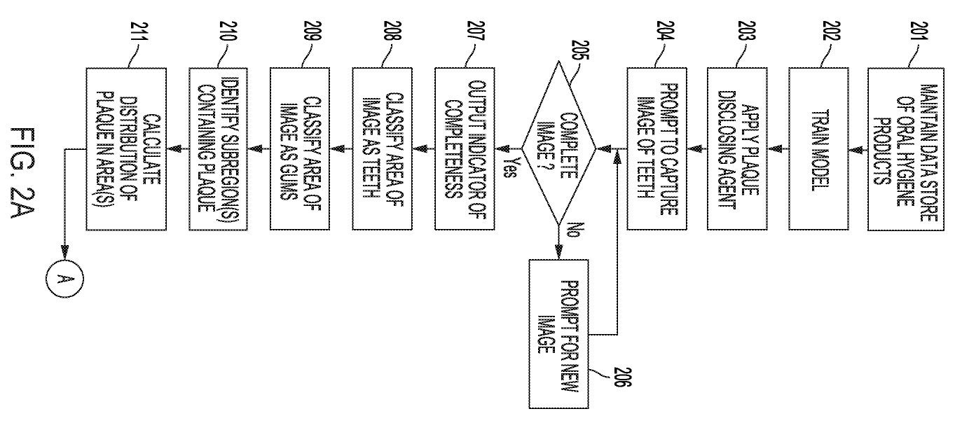

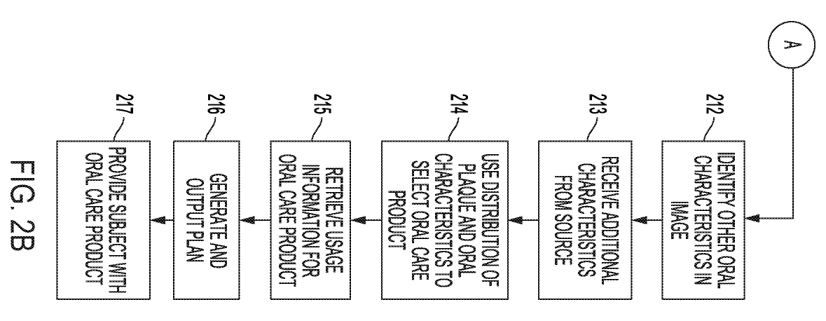

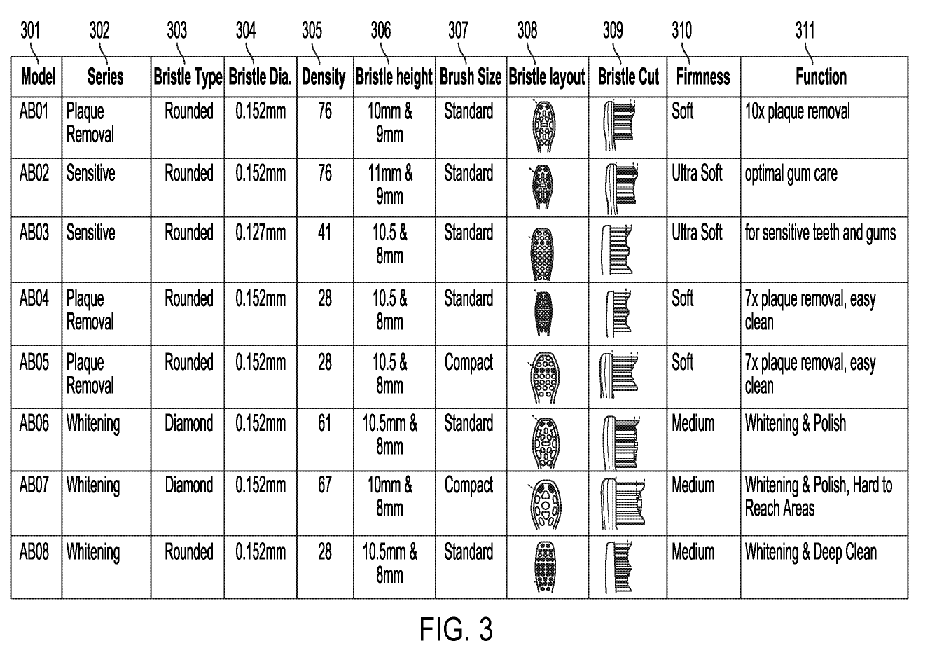

FIGS. 2A-2B are flowcharts with various steps that methods of selecting an oral hygiene product may include. As noted in FIG. 1 above, at 201 the system will maintain a data store with data for oral hygiene products. FIG. 3 illustrates, in tabular format, example descriptive information that the data store may hold, such as a product name or model number 301 and/or a code such as a universal product code (UPC), Amazon standard identification number (ASIN), or other identifying information. The descriptive information also may include information such as a product type or category 302 (such as toothbrush head, dental floss, or toothpaste, and/or a subcategory such as plaque removal, sensitive teeth or gums, etc.). The descriptive information also may include measurable parameters of the product itself, such as in the case of a toothbrush head bristle type 303, bristle diameter 304, bristle density 305, bristle height 306, brush head size 307, bristle layout 308, bristle cut 309, and bristle firmness 310. The descriptive information also may include a product function 311 for the product that represents an oral hygiene function for which the product was designed, such as plaque removal, gum care, or teeth whitening.

In some embodiments, the system may use a trained machine learning model to process image data and to classify areas of the image as corresponding to one or more dental conditions. In embodiments that do this, the method may include training the model at 202 by providing the model with a set of images of human mouths and/or teeth with labels identifying regions of each image that include various dental characteristics or images. For example, some image regions may be labeled as containing teeth, gums, the tongue, and/or other parts of the person’s mouth. Regions of some images may be labeled as showing one or more dental conditions, such as:

plaque (with or without being revealed by a plaque disclosing agent);

caries (i.e., cavities or tooth decay);

dental restorations;

occlusions in teeth;

surface deposits such as calculus or tartar;

demineralization zones in tooth enamel;

tooth stains (such as from coffee, wine, or tobacco);

specific parts of a tooth, such as the cusp;

areas in which saliva is present vs. areas that are dry; and/or

gum morphology, such as recession depth, papilla height, and gingival margin.

In some embodiments, the machine learning model may be a neural network, such as a convolutional neural network (CNN), that includes multiple layers of interconnected nodes. During training, the neural network learns to extract hierarchical features from the labeled training images. Earlier layers of the network may learn to detect low-level features such as edges, textures, and color gradients, while deeper layers may learn to detect higher-level features such as shapes corresponding to teeth, gums, plaque regions, or other dental characteristics. The outputs of the intermediate layers of the neural network form an embedding, which is a compressed vector representation of the input image that encodes the learned features. The final layers of the neural network use the embedding to classify various regions of images that it receives after being trained.

At 203 the electronic device will output a visual and/or audio message that prompts the user to apply an indicator material such as a plaque disclosing agent to their teeth. This will prompt the user to insert a plaque disclosing agent into their mouth, and to hold the plaque disclosing agent in their mouth for a time period that is effective to cause the material to dye areas of plaque on their teeth. Many now or hereafter known plaque disclosing agents may be used in this step, and example materials include a non-toxic dye that is available in tablet, liquid, or powdered form. Example dyes include erythrosine, vegetable dyes such as Phloxine B that will temporarily stain areas of the person’s teeth that contain plaque. The person may be prompted to chew the material (if in tablet form), or swish a solution containing the material, and hold it in their mouth for a period of time, such as at least one minute. After that period of time, the person may spit out any residue that has not attached to the person’s teeth.

At 204 the system will prompt the person or other user to capture images of the person’s teeth, and in response the system will receive image data that includes one or more images of the person’s teeth. The person may be a patient of a dental professional, or a consumer who is searching for an oral hygiene product that is suitable for their dental condition. The images may include images of the patient’s front teeth 107 and will sufficiently show the teeth so that the boundaries of the frontmost teeth are fully visible in the picture. Optionally, the image or images of either of the patient’s arches may be a composite of multiple images captured from a camera that was in multiple positions, such as an intraoral scanning device that captures images as it is moved within the person’s mouth. This document may use the term “image data” to generally refer to a single image, multiple images and/or a composite image, depending on what is available for the system to process.

At 205 the system will parse the image data to determine whether it is a complete image. If the image is not a complete image (205: NO), it will prompt the user to capture a new image at 206. If the image is complete (205: YES), the system may output an indicator of completeness at 207. To determine whether an image is complete, the system may use any suitable image processing algorithm or to identify the patient’s teeth and other oral structures. The system may use an intelligent image classification model that has been trained using labeled image data of teeth and other items, such as those available using the TensorFlow library, the OpenCV image processing algorithms, or Caffe. The system also may use an edge detection algorithm to determine whether complete borders are present for at least a threshold number of teeth, and thus at least a threshold number of teeth are not occluded by the person’s lips or otherwise. For example, if the camera is a 3D intraoral scanning device, the system may prompt the user to move the device to different positions and/or angles within the person’s mouth. The system may cause the camera or the device that includes the camera to emit a yellow light, red light, blinking light, other color or pattern of light, or no light when the imaging process is not yet complete, thus prompting the user to continue scanning. When the system has received a group of images that collectively show a threshold number of teeth, the system may play a different sound, or it may emit light of a different color or pattern, to indicate completeness at 207. The system may generate a composite image using any now or hereafter known image stitching or other compositing technique.

At 208, once the system has received suitable image data, the system will process the image data to classify areas of the image that contain the teeth. The system may perform the classification using a machine learning image classification model that has been trained using labeled image data that identify segments that correspond to teeth, such as those described above. When using a machine learning model such as a convolutional neural network, the system may pass the image through multiple convolutional and pooling layers that extract features from the raw pixel data. These layers transform the pixel data into an embedding, which is a lower-dimensional vector representation that captures the salient features of the image. The embedding is then passed through one or more fully connected layers that learn decision boundaries between different classes. The final layer of the network may apply a softmax activation function or other function that outputs a probability distribution over the possible class labels, and the system may assign each region of the image to the class with the highest probability, or to all classes for which the probability exceeds a threshold. Alternatively, the system may use an image segmentation algorithm such as those described above to process the image and identify one or more segments that correspond to the teeth. Optionally, at 209 the system also may use the same or similar models or algorithms to classify areas of the image that contain soft tissue, and in particular gums (i.e., soft tissue adjacent to the teeth), in the person’s mouth.

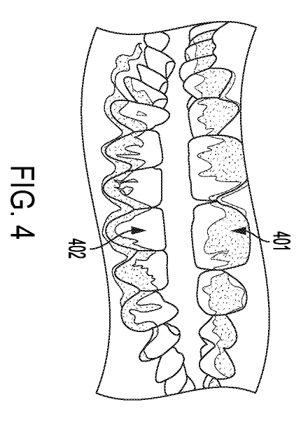

At 210 the system will classify one or more subregions of the image that display plaque on the person’s teeth and/or gums, and optionally other subregions such as the tongue. To identify the subregions, after the system has classified an area as teeth, gums, tongue, or other, the system may then process image pixels in that area to identify a number, percentage, or other measure of pixels that have a color value corresponding to that of the plaque disclosing agent. This is illustrated by way of example in FIG. 4, in which areas in which plaque appear 401 are relatively darker than areas in which plaque does not appear 402 in any significant amount. The system may determine that any pixels having a color value within a specified range of the plaque disclosing agent’s color value, or that exhibits color of at least a threshold intensity, is one on which plaque is present. At 211 the system will extract pixel data from the image and use the pixel data to calculate a distribution of the plaque in the areas or areas. For example, the system may extract pixel data to calculate a relative measurement of size (such as surface area, number of pixels, or other measurement of size) of the subregion in which plaque is present to the overall size of the area (using the same parameters of measurement. For example, the system may compare the number of pixels that make up an area that shows a particular tooth, an area with multiple teeth, and/or an area of gums to the number of pixels within that area that display plaque. The distribution of plaque in the area may be the ratio or percentage of pixels containing plaque to that of the overall area.

In some embodiments, the system may process the pixel data using one or more color space transformations to enhance plaque detection accuracy. For example, the system may convert image pixels from an RGB (red, green, blue) color space to an HSV (hue, saturation, value) or LAB color space, which may more effectively isolate the chromatic characteristics of the plaque disclosing agent from the natural tooth color. After color space transformation, the system may apply thresholding operations to segment pixels that fall within a predetermined hue or chromaticity range corresponding to the disclosing agent. The system may additionally perform morphological operations such as erosion and dilation to reduce noise and refine the boundaries of detected plaque regions.

In some embodiments, the system may analyze pixel intensity values within the classified tooth area to generate a plaque intensity map. The intensity map may assign a plaque severity score to each pixel or group of pixels based on the degree to which the pixel's color deviates from an expected baseline tooth color and approaches the color profile of the plaque disclosing agent. The system may then aggregate these pixel-level scores to produce regional plaque concentration metrics, such as identifying areas of heavy plaque accumulation versus areas of light plaque presence. This pixel-level analysis can enable the system to provide more precise plaque distribution data for product selection.

At 212 the system may identify one or more other oral characteristics in the image. For example, if any image shows plaque on the person’s teeth, the system may further process the image to identify: (a) whether the locations of the plaque on the teeth correspond to a cervical location (i.e., in the area adjacent to the gum line); (b) an occlusal location (i.e., the area where teeth come into contact with food when biting food); (c) a lingual location (i.e., the area proximal to the tongue); (d) an interproximal location (i.e., in the area that is adjacent to a neighboring tooth); and/or (e) a frontal location (i.e., between the interproximal locations, and below or above the cervical location). The system also may identify other characteristics such as gum recession.

As another example, at 212 the system also may use the trained model to identify the plaque and/or other oral characteristics of the teeth such as:

dentition (missing teeth);

caries (cavities or tooth decay);

dental restorations (such as crowns, implants, or dentures);

enamel integrity (such as demineralization zones, white spot lesions, enamel translucency and/or thickness indicators, surface roughness and/or microtexture);

surface deposits (such as calculus, tartar, or stains from tobacco, coffee, or wine);

wear patterns, attrition, and/or erosion;

orthodontic treatments, brackets, permanent retainers and/or other attachments;

occlusions;

tooth morphology such as shape and/or size of teeth, width, height, and/or curvature;

alignment and spacing (e.g., crowding, gaps, interdental gap width, tooth angulation, and/or tooth rotation);

cusp depth and/or fissure complexity on molars; and

other tooth characteristics.

The system also may use the trained model to identify other oral characteristics of the gums such as calculus, pockets, inflammation (as indicated by color gradients and/or swelling), bleeding, bone loss, gum recession, gingivitis, papilla height loss, gingival margin irregularity, and/or periodontitis.

The system also may use the trained model to identify other characteristics of other areas of the mouth, such as saliva and oral fluid characteristics (i.e., saliva flow, dry mouth indicators, or excessive salivation), saliva properties (such pH as estimated by color indicators, viscosity, or buffering capacity proxies); and/or characteristics of the tongue or other soft tissue.

In each of the cases above, the model will have been trained on images that include some or all of the features listed above, in which the features have been labeled so that the model learns to recognize patterns associated with each label. During training, the model adjusts internal parameters (weights) to minimize the difference between its predicted classifications and the actual labels in the training data. When receiving a new image, the system will extract pixel data from the image (such as color properties, location, and neighboring pixel data), pass the pixel data through the trained neural network layers to generate an embedding, and then process the embedding through the classification layers of the network. The classification layers apply learned decision boundaries to the embedding to predict which label or labels correspond to each region of the image. The model outputs classification scores or probabilities for each possible label, and the system assigns each pixel or region to the label with the highest score, or to all labels for which the score exceeds a confidence threshold.

For example, the system may process the image to identify areas of tartar on the person’s teeth. Plaque is a soft biofilm that can be disclosed (i.e., made apparent) when contacted with a disclosing agent. Tartar (also known as calculus) is a hardened form of plaque, typically yellow in color, which is thus visible even without a disclosing agent and thus does not need to be disclosed. The system may detect tartar using image processing algorithms, such as those that identify areas of shading or color that correspond to expected locations of tartar.

At 213 the system may receive other oral characteristics of the person from a data source, such as a patient profile data set and/or information that is received in response to questions or other prompts. For example, the system may access a patient profile data set that includes a dental history to determine what dental restorations, orthodontic treatments, or previous conditions are reflected in the record. As another example, the system may provide, via a chatbot function, questions that prompt the patient to provide information that describes the one or more oral characteristics. The chatbot function may be implemented using an artificial intelligence model, such as a transformer model. The system also may receive information relating to an arch shape of the person, which may be determined using methods such as those described in U.S. Patent Application Publication No. 2024/0338929, the disclosure of which is incorporated into this document by reference.

Optionally, at step 213 the system also may prompt the user to provide a history of the person’s food intake over a period of time. The system may receive this information in response to questions presented to the user. Alternatively, the person may capture images of food before eating it over the period of time, and the system may process the images in an artificial intelligence model that has been trained in object recognition to label the food and/or attributes of the food that appear in the image. The system may then use this information to update the patient’s profile and assess characteristics of the food that the person eats. The system may then associate oral characteristics that relate to that food with the patient. For example, if the person’s food intake indicates that the person regularly eats sugary and/or sticky food, then the system may classify the person as having a high risk of cavities, and it may consider that high risk to be an oral characteristic of the person.

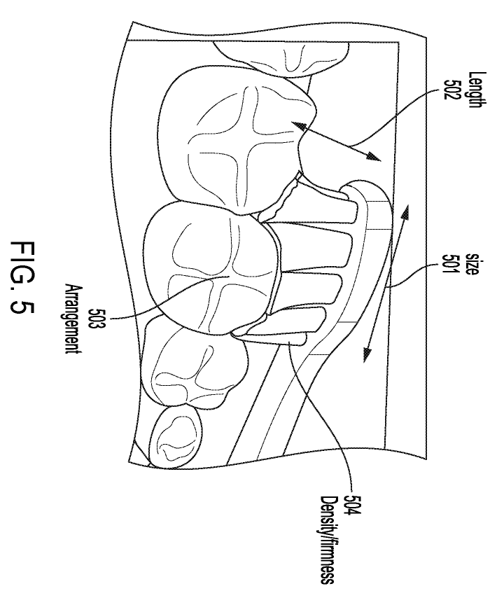

At 214 the system will use the measured plaque distribution and at least some of the person’s oral characteristics to select, from the data store, an oral care product that corresponds to the distribution of plaque and the one or more oral characteristics. For example, with reference to FIG. 5, the system may identify the size and shape of one or more of the person’s teeth and select a brush head having a size 501 and length 502 that corresponds to the person’s tooth size and shape. The system also may only consider products having an associated product function that corresponds to the oral characteristics (such as plaque removal if the teeth exhibit extensive plaque). The system may then select, from products meeting the size, length, product function, and/or other requirements, a brush head having a bristle arrangement (i.e., layout or pattern) 503 and density and/or firmness 504 that is appropriate for the plaque distribution. The system also may use the characteristics to further narrow and/or expand the field of candidate products for selection, such as by identifying a brush head having a function that corresponds to one or more of the characteristics.

Optionally, the system may use some of the additional information to eliminate one or more categories from consideration. For example, if the person is an adult, the system may eliminate all categories that are designed for individuals whose age status is that of a child; if the subject is a child the system may eliminate all categories that are designed for individuals whose age status is that of an adult. In addition, some dental conditions may take priority over others. For example, if the patient has braces, the system may select a brush head category that is appropriate for braces, before it considers the patient’s plaque distribution or other characteristics.

Optionally, the system may present the user with multiple candidate products meeting the criteria described above, and the user may select one or more of the products. Alternatively, the system may present the user with a single product, which the user may accept or reject.

Optionally, if the oral characteristics include food intake habits (as described above), the system may use that information and the person’s current plaque distribution to predict a future plaque distribution for the person’s teeth at a future time. The system may do this by sending the patient’s plaque distribution and food intake habits to an AI model that has been trained on images showing plaque distribution progression, and the AI model may use this information to predict how the patient’s plaque distribution will progress over a period of time. If so, then when selecting an oral care product, the system may use not only the person’s current plaque distribution, but also (or alternatively) the person’s predicted future plaque distribution to select the oral care product.

Additional oral characteristics that the system may collect, and methods of collecting and using such characteristics, are disclosed in U.S. Patent Application Publication Number 2024/0338929, “Method and System for Selecting Oral Hygiene Tool”, the disclosure of which is incorporated into this document by reference.

When the system presents, or when the user selects, an oral care product, at 215 the system may retrieve usage information such as instructions for use. The system may then present that information to the user, along with the identification of the product, as a report that serves as a personalized oral hygiene plan 216 for the user’s oral hygiene. The personalized plan may include information such as: recommendations for oral hygiene products for the person to use to clean and maintain their teeth, or for a medical professional to use when treating the person’s teeth; instructions for use of such products; recommendations for changes in habits that can affect oral hygiene, such as eating habits; and other information.

At 217 the system will provide the person or other subject with the selected oral care product. The system may do this as part of an online tool that helps the person select and place an order for a toothbrush (i.e., a manual toothbrush or a brush head for an electric toothbrush) that corresponds to the person’s dental condition. For example, the system may generate an electronic message, or output via a user interface, information identifying the selected oral care product. The user interface may include a display and input device such as a microphone, keyboard or touch pad. Alternatively or in addition, the user interface may include a smart speaker that may output the information via an audio prompt. The user interface may include or be associated with an electronic shopping cart that is part of an e-commerce platform. The subject may select or approve placement of the identified oral care product into the shopping cart, and then use the shopping cart to order the toothbrush. Alternatively, the system may automatically cause the e-commerce platform to order the oral care product without requiring the person to actively place the order. Alternatively, the system may implement step 217 by providing a recommendation with information about the toothbrush to the subject’s dental professional, who will provide or arrange to provide the subject with the selected brush as part of a course of treating the subject’s dental condition as a patient of the dental professional. Other methods to provide the subject with the selected oral care product may be used.

Optionally, before providing the subject with the selected oral care product , the system may give the subject or another user the option to choose from one or more available styles for the oral care product, such as bristle colors or patterns. Then, when providing the subject with the selected oral care product at 217, the system will provide the subject with the selected product in the selected style.

Optionally, when presenting the subject with an oral care product, the system also may provide the subject with information about an additional oral care product that should be used with the primary product. For example, if the system presents the person with a recommendation for a toothbrush, it also may recommend a toothpaste having a level of abrasiveness or other characteristic that corresponds to one or more of the person’s oral characteristics. As another example, the system may recommend an interdental cleaning device, such as dental floss, an interdental brush, a water flosser, etc. As another example, the system may recommend another oral care product such as mouthwash, a tongue scraper, or teeth whitening strips. The system may select from multiple options of these products based on the imaged information about the person’s teeth. For example, if the images show that the person has teeth that exhibit crowding, the system may select a relatively narrower floss from a group of candidate flosses and recommend the relatively narrower floss to the person.

Optionally, the features and functions described above may be implemented in a software application that can be run on a computing device. The application may include other features, such as a shopping platform by which a user can purchase the recommended oral care products. The application also may include other functions, such as:

a camera application that guides the user to use a camera of the electronic device to capture a complete image of the person’s teeth; or

a video player that can output a video containing the usage instructions and/or the oral hygiene plan.

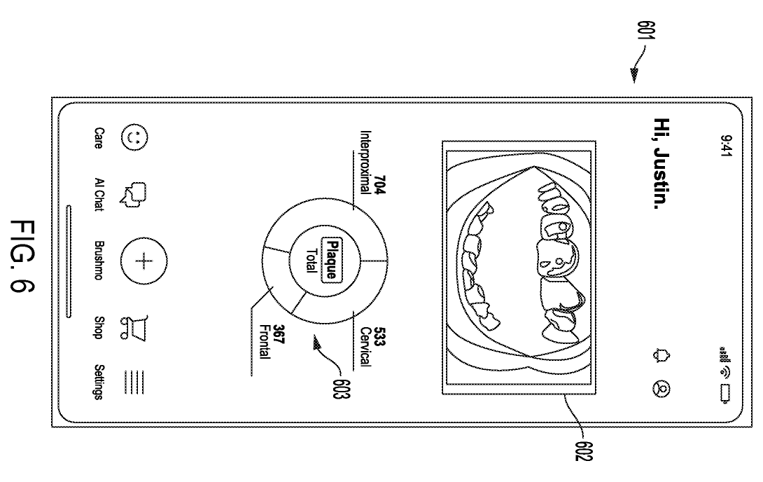

FIG. 6 depicts an example user interface 601 of such an application. The user interface 601 is output on a display device of an electronic device. The user interface 601 includes an embedded video player 602 that depicts, optionally in real time as images are captured, and/or after the images are captured, the latest image (or image composite) of the person’s teeth. In this case, the teeth displayed in the video have a plaque disclosing agent applied to them, thus revealing areas containing plaque as dark areas on the person’s teeth. The application may enhance the image by adding boundaries around the person’s individual teeth as received from the image segmentation algorithm, and/or by enhancing the intensity and/or other characteristics of pixels in areas that contain plaque. The system also may display a graphic representation of the measured plaque distribution 603, optionally as further sub-distributed in areas such as interproximal, cervical, and frontal, as calculated in real-time by the system.

The application also may provide the user with other features resulting from image analysis. For example, the system may enable the user to capture an image a used toothbrush head, and the system may send that image to an AI model that has been trained to identify characteristics of the brush to determine whether the brush exhibits characteristics showing that it should be replaced. For example, if the model classifies the brush head as having frayed and uneven bristles, or flattened bristles, the system may output a message indicating that the toothbrush should be replaced.

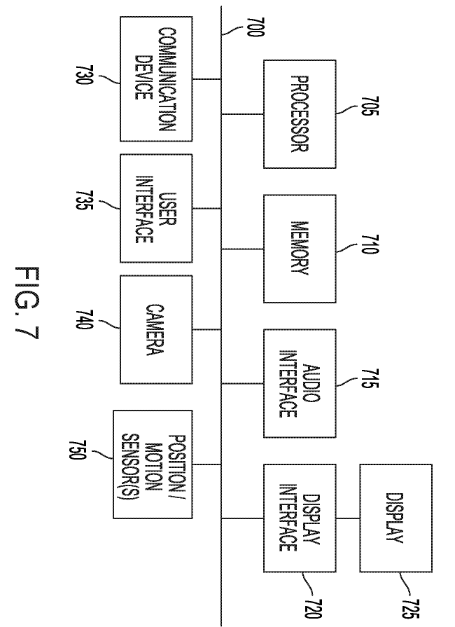

FIG. 7 depicts example components that may be included in any of the electronic devices of the system, such as a smartphone, a tablet computing device, or a local or remote computing device in the system. A conductive path such as a bus 700 serves as a communication path via which messages, instructions, data, or other information may be shared among the other illustrated components of the hardware. Processor 705 is a central processing device of the system, configured to perform calculations and logic operations required to execute programming instructions. As used in this document and in the claims, the terms “processor” and “processing device” may refer to a single processor or any number of processors in a set of processors that collectively perform a set of operations, such as a central processing unit (CPU), a graphics processing unit (GPU), a remote server, or a combination of these. Read only memory (ROM), random access memory (RAM), flash memory, hard drives and other devices capable of storing electronic data constitute examples of memory devices 710. A memory device may include a single device or a collection of devices across which data and/or instructions are stored.

An optional display interface 720 may enable information to be displayed on a display device 725 in visual, graphic or alphanumeric format. An audio interface 715 with audio output (such as a speaker) also may be provided. Communication with external devices may occur using various communication devices 730 such as a wireless antenna, a radio frequency identification (RFID) tag and/or short-range or near-field communication transceiver, each of which may optionally communicatively connect with other components of the device via one or more communication systems. The communication device 730 may be configured to be communicatively connected to a communications network, such as the Internet, a local area network or a cellular telephone data network.

The hardware may also include a user interface device 735 that includes one or more input devices that can receive data and/or commands from a user. Example user interface devices 735 include a keyboard, a mouse, touchscreen, a touch pad, a remote control, a pointing device, and/or a microphone. A camera 740 may include image sensors and other hardware that can capture video and/or still images. The system also may include one or more positional and/or motion sensors 750 that can detect position and movement of the device. Examples of motion sensors include gyroscopes, accelerometers, and inertial measurement units (IMUs). Examples of positional sensors include a global positioning system (GPS) sensor device that receives positional data from an external GPS network.

Based on the teachings contained in this disclosure, it will be apparent to persons skilled in the relevant art(s) how to make and use embodiments of this disclosure using data processing devices, computer systems and/or computer architectures other than that shown in FIG. 6. In particular, embodiments can operate with software, hardware, and/or operating system implementations other than those described in this document.

The following paragraphs provide additional information about various terms used in this document:

In this document, when terms such as “first” and “second” are used to modify a noun, such use is simply intended to distinguish one item from another and is not intended to require a sequential order unless specifically stated.

The term “approximately” when used in connection with a numeric value, is intended to include values that are close to, but not exactly, the number. For example, in some embodiments, the term “approximately” may include values that are within +/- 10 percent (or, in some embodiments, +/- 5 percent, +/- 3 percent, or +/- 1 percent) of the value. By way of example, the phrase “approximately 100%” will include values from 90% to 100%, and the phrase “approximately 90 degrees” will include values from 81 degrees to 99 degrees.

The term “substantially,” when used in connection with a value, is intended to mean approximately, within a threshold tolerance that is a percentage corresponding to any of the percentages described in the previous paragraph. For example, items described as “substantially the same,” “substantially equal,” or “substantially planar,” may be exactly the same, equal, or planar, or may be the same, equal, or planar within acceptable variations that may occur, for example, due to manufacturing processes and/or tolerances.

An “electronic device” or a “computing device” refers to a device or system that includes a processor and memory. Each device may have its own processor and/or memory, or the processor and/or memory may be shared with other devices as in a virtual machine or container arrangement. The memory will contain or receive programming instructions that, when executed by the processor, cause the electronic device to perform one or more operations according to the programming instructions. Examples of electronic devices include personal computers, servers, mainframes, virtual machines, containers, gaming systems, televisions, digital home assistants and mobile electronic devices such as smartphones, fitness tracking devices, wearable virtual or augmented reality devices, Internet-connected wearables such as smart watches and smart eyewear, personal digital assistants, cameras, tablet computers, laptop computers, media players and the like. Electronic devices also may include appliances and other devices that can communicate in an Internet-of-things arrangement, such as smart thermostats, refrigerators, connected light bulbs and other devices. Electronic devices also may include components of vehicles such as dashboard entertainment and navigation systems, as well as on-board vehicle diagnostic and operation systems. In a client-server arrangement, the client device and the server are electronic devices, in which the server contains instructions and/or data that the client device accesses via one or more communications links in one or more communications networks. In a virtual machine arrangement, a server may be an electronic device, and each virtual machine or container also may be considered an electronic device. In the discussion above, a client device, server device, virtual machine or container may be referred to simply as a “device” for brevity. Additional elements that may be included in electronic devices are discussed above in the context of FIG. 7.

The terms “processor” and “controller” refer to electronic device hardware that is configured to execute programming instructions. The terms “processor” and “controller” may refer to either a single processor or controller, or to multiple processors or controllers that together implement various steps of a process. Unless the context specifically states that a single processor or controller is required or that multiple processors or controllers are required, the terms “processor” and “controller” include both the singular and plural embodiments.

The terms “memory,” “memory device,” “computer-readable medium” and “data store” each refer to a non-transitory device on which computer-readable data, programming instructions or both are stored. A “computer program product” combination of a memory device and the programming instructions stored in it. Unless the context specifically states that a single device is required or that multiple devices are required, the terms defined in this paragraph include both the singular and plural embodiments, as well as portions of such devices such as memory sectors.

In this document, the terms “camera” and “imaging device” generally refer to device having a hardware sensor that is configured to acquire digital images. A camera may capture still and/or video images, and it optionally may be used for other imagery-related applications. For example, a camera may be a user-controllable device such as a DSLR (digital single lens reflex) camera, mobile phone camera, or video camera. The camera may be part of an image capturing system that includes other hardware components, such as a 3D dental scanning device, which is also known as an intraoral scanning device. Imaging devices also may include those that capture other types of images, such as fluorescence imaging devices, infrared imaging devices, and LIDAR systems.

The term “classifier” means an automated process by which an artificial intelligence system may assign a label or category to one or more data points. A classifier includes an algorithm that is trained via an automated process such as machine learning. A classifier typically starts with a set of labeled or unlabeled training data and applies one or more algorithms to detect one or more features and/or patterns within data that correspond to various labels or classes. The algorithms may include, without limitation, those as simple as decision trees, as complex as Naïve Bayes classification, and/or intermediate algorithms such as k-nearest neighbor. Classifiers may include artificial neural networks (ANNs), support vector machine classifiers, and/or any of a host of different types of classifiers. Once trained, the classifier may then classify new data points using the knowledge base that it learned during training. The process of training a classifier can evolve over time, as classifiers may be periodically trained on updated data, and they may learn from being provided information about data that they may have mis-classified. A classifier will be implemented by a processor executing programming instructions, and it may operate on large data sets such as image data, LIDAR system data, and/or other data.

The terms “artificial intelligence model” and “AI model” refer to system of software and hardware that can generate output in response to one or more prompts, other input, or other action without being explicitly programmed or using a rules-based structure to generate its output. Instead, a model learns to generate output in a training process, which can use actual results of the real-world process that is being modeled. Such systems or models are understood to be necessarily rooted in computer technology, and in fact, cannot be implemented or even exist in the absence of computing technology. While machine learning systems utilize various types of statistical analyses, machine learning systems are distinguished from statistical analyses by virtue of the ability to learn without explicit programming and being rooted in computer technology. Examples of AI models include those known embodied in neural networks such as convolutional neural networks (“CNNs”).

“Training” of an AI model may include building and/or updating a machine learning model from a sample dataset (referred to as a “training set”), evaluating the model against one or more additional sample datasets (referred to as a “validation set” and/or a “test set”) to decide whether to keep the model and to benchmark how good the model is, and using the model in a production environment to make predictions or decisions, or to generate content, based on new input data. Training an AI model may include supervised learning, in which the training data is labeled with the correct output so that the model learns the correct output, as well as unsupervised learning in which the model receives unlabeled data and discovers patterns and insights without explicit human instruction.

The features and functions described above, as well as alternatives, may be combined into many other different systems or applications. Various alternatives, modifications, variations or improvements may be made by those skilled in the art, each of which is also intended to be encompassed by the disclosed embodiments.

As described above, this document discloses system, method, and computer program product embodiments for identifying dental care products. The system embodiments include a local computing device, which may have access to one or more remote computing devices. In some embodiments, one or more of the remote computing devices also may be part of the system. The computer program embodiments include programming instructions, stored in a memory device, that are configured to cause a processor to perform the methods described in this document.

Without excluding further possible embodiments, certain example embodiments are summarized in the following clauses:

Clause 1. A method for developing an individualized oral hygiene plan for a person, the method comprising: (a) maintaining a data store comprising descriptive information for a plurality of oral care products; (b) causing an electronic device to output a prompt to capture an image of teeth of a person; (c) in response to the prompt, receiving the image; (d) processing the image to determine an oral condition of the person by classifying an area of the image as corresponding to the teeth, automatically identifying a subregion of the area in which plaque is present, and extracting pixel data from the image and using the extracted pixel data to calculate a distribution of plaque on the teeth; (e) receiving one or more oral characteristics of the person; (f) based on the distribution of plaque and the one or more oral characteristics, selecting, from the data store, an oral care product that corresponds to the distribution of plaque and the one or more oral characteristics; and (g) generating and outputting an oral hygiene plan comprising an identification of the selected oral care product.

Clause 2. The method of clause 1, wherein using the extracted pixel data to calculate the distribution of plaque comprises: measuring a first surface area corresponding to the area of the image; measuring a second surface area corresponding to segments of the subregion in which the plaque is present; and calculating e a ratio or percentage for the second surface area with respect to the first using relative surface area.

Clause 3. The method of clause 1 or 2, wherein processing the image to determine the oral condition of the person further comprises: (a) classifying an additional area of the image as corresponding to gums; (b) automatically identifying a subregion of the additional area in which plaque is present on the gums; and (c) using relative measurements of the additional area and the subregion of the additional area to calculate a distribution of plaque on the gums.

Clause 4. The method of any of clauses 1-3, wherein causing the electronic device to output the prompt comprises: (a) prompting the person to hold a plaque disclosing agent in their mouth for a period of time that is effective to cause the plaque disclosing agent to dye areas of plaque on the teeth; and (b) prompting a user to, after the person holds the plaque disclosing agent in the person’s mouth for the period of time, use a camera to capture the image.

Clause 5. The method of any preceding clause, wherein causing the electronic device to output the prompt comprises: (a) prompting the person to use an imaging device capture a preliminary image of the teeth; (b) processing the image to determine whether the image contains a complete representation of the teeth; and (c) outputting a visual and/or audio indicator of completeness if the image contains the complete representation of the teeth, otherwise prompting the user to reposition the imaging device and/or the teeth to capture an additional image of the teeth.

Clause 6. The method of any preceding clause, wherein classifying the area of the image as corresponding to one or more of the teeth comprises: (a) using an image segmentation algorithm to process the image and identify one or more segments that correspond to the teeth; or (b) providing the image to an artificial intelligence model that has been trained to segment and apply labels to teeth in images.

Clause 7. The method of clause 6, wherein: (a) processing the image to assess the oral condition of the person further comprises identifying whether a location of the plaque on the teeth corresponds to a cervical location, an interproximal location, and/or a frontal location; and (b) selecting the oral care product is also based on the identified location.

Clause 8. The method of any preceding clause, further comprising training an artificial intelligence model to segment and apply labels to teeth in images, and wherein classifying the area of the image as corresponding to the teeth comprises: (a) providing the image to the artificial intelligence model, and (b) receiving, from the artificial intelligence model, a segmented image with labels associated with one or more segments of the segmented image.

Clause 9. The method of any preceding clause, wherein receiving the one or more oral characteristics of the person comprises providing, via a chatbot function, questions that prompt the patient to provide information that describes the one or more oral characteristics.

Clause 10. The method of any preceding clause, wherein receiving the one or more oral characteristics of the person comprises accessing a patient profile comprising a dental history for the person.

Clause 11. The method of any preceding clause, wherein: (a) the descriptive information for a plurality of oral care products comprises, for each of the oral care products, one or more product functions; and (b) selecting, from the data store, the oral care product that corresponds to the distribution of plaque and the one or more oral characteristics comprises selecting an oral care product with one or more product functions that are associated with the distribution of plaque and/or the one or more oral characteristics.

Clause 12. The method of any preceding clause, wherein: (a) processing the image further comprises identifying locations of soft tissue adjacent to the teeth and identifying areas of plaque in the soft tissue; and (b) selecting, from the data store, the oral care product comprises identifying a toothbrush having a brush head shape or size that corresponds to the areas of plaque in soft tissue.

Clause 13. The method of any preceding clause, wherein: (a) processing the image data comprises identifying a shape of the teeth; and (b) selecting, from the data store, the oral care product comprises identifying a toothbrush having a brush head shape that corresponds to the identified shape of one or more of the teeth.

Clause 14. The method of any preceding clause, wherein: (a) processing the image data comprises identifying a size of one or more of the teeth; and (b) selecting, from the data store, the oral care product comprises identifying a toothbrush model having a brush head size that corresponds to the identified size of the one or more of the teeth.

Clause 15. The method of any preceding clause, wherein: (a) processing the image to classify the teeth comprises identifying locations of soft tissue adjacent to the teeth, identifying locations of plaque in the locations of soft tissue, and identifying a shape or size of one or more of the teeth; and (b) selecting, from the data store, the oral care product comprises identifying a toothbrush model that has (i) a brush head shape, stiffness, or both that corresponds to the locations of plaque, and (ii) a brush head shape or size that corresponds to the identified shape or size of the one or more of the teeth.

Clause 16. The method of any preceding clause further comprising, in response to receiving to a user acceptance of the selected oral care product, also outputting information about an additional oral care product.

Clause 17. The method of any preceding clause further comprising outputting, on a display device, a user interface that includes one or more of the following: (i) a video player that depicts, in real time, an enhanced image that shows the distribution of plaque; or (ii) a graphic representation of the distribution of plaque on the teeth.

Clause 18. The method of any preceding clause, further comprising providing the person with the selected oral care product.

Clause 19. The method any preceding clause, further comprising: (a) retrieving, from the data store, usage information for the selected oral care product; and (b) when generating and outputting the oral hygiene plan, including the usage information in the oral hygiene plan.

Clause 20. A computer program product comprising a memory containing programming instructions that are configured to cause a processor to implement a method corresponding to any preceding clause.

Clause 21. A system comprising a processing device, and a memory containing programming instructions that are configured to cause a processor to implement a method corresponding to any preceding clause.

Clause 22. The system of clause 21, further comprising a display device.

Clause 23. The system of clause 21 or 22, further comprising an intraoral scanning device or another type of camera.

Clause 24: The system of clause 21, further comprising: (a) a data store comprising descriptive information for a plurality of oral care products; (b) a plaque disclosing agent; (c) a camera; and (d) a memory containing programming instructions that, when executed, will cause a processor to implement the method.

1. A method for developing an individualized oral hygiene plan for a person, the method comprising:
maintaining a data store comprising descriptive information for a plurality of oral care products;
causing an electronic device to output a prompt to capture an image of teeth of a person;
in response to the prompt, receiving the image;
processing the image to determine an oral condition of the person by:
classifying an area of the image as corresponding to the teeth,
automatically identifying a subregion of the area in which plaque is present, and
extracting pixel data from the image and using the extracted pixel data to calculate a distribution of plaque on the teeth in the area;
receiving one or more oral characteristics of the person;
based on the distribution of plaque and the one or more oral characteristics, selecting, from the data store:
an oral care product that corresponds to the distribution of plaque and the one or more oral characteristics, and
usage information for the selected oral care product; and
generating and outputting an oral hygiene plan comprising an identification of the selected oral care product and the usage information.

2. The method of claim 1, wherein using the extracted pixel data to calculate the distribution of plaque comprises:
measuring a first surface area corresponding to the area of the image;
measuring a second surface area corresponding to segments of the subregion in which the plaque is present; and
calculating a ratio or percentage for the second surface area with respect to the first surface area.

3. The method of claim 1, wherein processing the image to determine the oral condition of the person further comprises:
classifying an additional area of the image as corresponding to gums of the person;
automatically identifying a subregion of the additional area in which plaque is present on the gums; and
using relative measurements of the additional area and the subregion of the additional area to calculate a distribution of plaque on the gums.

4. The method of claim 1, wherein causing the electronic device to output the prompt comprises:
prompting the person to hold a plaque disclosing agent in their mouth for a period of time that is effective to cause the plaque disclosing agent to dye areas of plaque on the teeth; and
prompting a user to, after the person holds the plaque disclosing agent in the person’s mouth for the period of time, use a camera to capture the image.

5. The method of claim 1, wherein causing the electronic device to output the prompt comprises:
prompting the person to use an imaging device capture a preliminary image of the teeth;
processing the image to determine whether the image contains a complete representation of the teeth; and
outputting a visual and/or audio indicator of completeness if the image contains the complete representation of the teeth, otherwise prompting the person to reposition the imaging device and/or the teeth to capture an additional image of the teeth.

6. The method of claim 1, wherein classifying the area of the image as corresponding to one or more of the teeth comprises:
using an image segmentation algorithm to process the image and identify one or more segments that correspond to the teeth; or
providing the image to an artificial intelligence model that has been trained to segment and apply labels to teeth in images.

7. The method of claim 6, wherein:
processing the image to assess the oral condition of the person further comprises identifying whether a location of the plaque on the teeth corresponds to a cervical location, an interproximal location, and/or a frontal location; and
selecting the oral care product is also based on the identified location.

8. The method of claim 1, further comprising:
training an artificial intelligence model to segment and apply labels to teeth in images,
wherein classifying the area of the image as corresponding to one or more of the teeth comprises:
providing the image to the artificial intelligence model, and
receiving, from the artificial intelligence model, a segmented image with labels associated with one or more segments of the segmented image.

9. The method of claim 1, wherein receiving the one or more oral characteristics of the person comprises providing, via a chatbot function, questions that prompt the patient to provide information that describes the one or more oral characteristics.

10. The method of claim 1, wherein receiving the one or more oral characteristics of the person comprises accessing a patient profile comprising a dental history for the person.

11. The method of claim 1, wherein:
the descriptive information for a plurality of oral care products comprises, for each of the oral care products, one or more product functions; and
selecting, from the data store, the oral care product that corresponds to the distribution of plaque and the one or more oral characteristics comprises selecting an oral care product with one or more product functions that are associated with the distribution of plaque and/or the one or more oral characteristics.

12. The method of claim 1, wherein:
processing the image further comprises:
identifying locations of soft tissue adjacent to the teeth, and
identifying areas of plaque in the soft tissue; and
selecting, from the data store, the oral care product comprises identifying a toothbrush having a brush head shape or size that corresponds to the areas of plaque in soft tissue.

13. The method of claim 1, wherein:
processing the image data comprises identifying a shape of one or more of the teeth; and
selecting, from the data store, the oral care product comprises identifying a toothbrush having a brush head shape that corresponds to the identified shape of one or more of the teeth.

14. The method of claim 1, wherein:
processing the image data comprises identifying a size of the teeth; and
selecting, from the data store, the oral care product comprises identifying a toothbrush model having a brush head size that corresponds to the identified size of one or more of the teeth.

15. The method of claim 1, wherein:
processing the image to classify the teeth comprises:
identifying locations of soft tissue adjacent to the teeth,
identifying locations of plaque in the locations of soft tissue, and
identifying a shape or size of one or more of the teeth; and
selecting, from the data store, the oral care product comprises identifying a toothbrush model that has:
a brush head shape, stiffness, or both that corresponds to the locations of plaque, and
brush head shape or size that corresponds to the shape or size of the one or more of the teeth.

16. The method of claim 1, further comprising, in response to receiving a user acceptance of the selected oral care product, also outputting information about an additional oral care product.

17. The method of claim 1, further comprising outputting, on a display device, a user interface that includes one or more of the following:
a video that depicts, in real time, an enhanced image that shows the distribution of plaque; or
a graphic representation of the distribution of plaque on the teeth.

18. The method of claim 1, further comprising providing the person with the oral care product.

19. The method of claim 1, further comprising:
retrieving, from the data store, usage information for the selected oral care product; and
when generating and outputting the oral hygiene plan, including the usage information in the oral hygiene plan.

20. A system for providing an individualized oral hygiene plan to a person, the system comprising:
a data store comprising descriptive information for a plurality of oral care products;
a plaque disclosing agent;
a camera; and
a memory containing programming instructions that, when executed, will cause a processor to:
cause an electronic device to prompt application of the plaque disclosing agent to a person’s teeth, and then to use of the camera to capture an image of the teeth, and
in response to receiving the image:
process the image to determine an oral condition of the person by:
classifying an area of the image as corresponding to the teeth;
automatically identifying a subregion of the area in which plaque is present; and
extracting pixel data from the image and using the extracted pixel data to calculate a distribution of plaque on the teeth in the area;
based on the distribution of plaque and one or more oral characteristics of the person, select, from the data store, an oral care product that corresponds to the distribution of plaque and the one or more oral characteristics; and
generate and output an oral hygiene plan comprising an identification of the selected oral care product.
